(19) United States
(12) Patent Application Publication
(10) Pub. No.: US 2002/0019007 A1
(43) Pub. Date: Feb. 14, 2002

(54) PCR methods and materials

(76) Inventor: Wayne A. Jensen, Wellington, CO (US)

Correspondence Address: HESKA CORPORATION, INTELLECTUAL PROPERTY DEPT., 1613 PROSPECT PARKWAY, FORT COLLINS, CO, 80525, US

(21) Appl. No.: 09/927,483

(22) Filed: Aug. 10, 2001

Related U.S. Application Data
(63) Continuation-in-part of application No. 09/398,179, filed on Sep. 17, 1999, now Pat. No. 6,300,072.
(60) Provisional application No. 60/101,023, filed on Sep. 18, 1998.

(51) Int. Cl. C12Q 1/68; C07H 21/04; C12P 19/34; C12N 1/21; C12N 15/74

(52) U.S. Cl. 435/6; 435/91.2; 435/252.3; 435/320.1; 536/23.7

(57) ABSTRACT

In broad terms, the present invention includes materials and methods useful to distinguish between and among species of a genus. The present methods utilize the differences in PCR amplicon sizes to specifically identify a given species.

### BACKGROUND OF THE INVENTION

The present invention is concerned with speciation of organisms, for the purpose of improving differential diagnosis of disease. The assays currently available to distinguish between or among species have not always met the expectations of consumers because they are either too costly, cumbersome or unavailable.

Polymerase chain reaction (PCR) and serological assays are currently used to distinguish among species. Serological tests present problems with cross-reactivity and available PCR tests are complicated. Typically, PCR-based assays require three steps: 1) conducting PCR using a primer set which distinguishes among members of different genera, but not among members of the same genus; 2) digesting the PCR products with restriction enzymes and 3) distinguishing among species on the basis of restriction digest patterns. One assay uses several sets of species-specific primers instead of digestion with restriction enzymes, with identification of the PCR products made by size. Minnick and Barbian, 31 J Microb Meth 51 (1997).

One genus of microorganisms, Bartonella, causes a variety of species-dependent disease states in humans, and is therefore important to speciate prior to treatment. Humans infected with bacteria from the genus Bartonella display a variety of pathogies, and appropriate treatment has been surmised as dependant on the species causing the pathology. For instance, the species B. henselae (relatively common in flea-infested areas) presents as cat scratch disease or atypical cat scratch disease, and health care professionals continue to debate the appropriate antibiotic treatment. Bass et al., 16 Pediatr. Infect. Dis. J., 163 (1997). B. clarridgeiae, another causative agent of cat scratch disease, can be treated with antibiotics, but it is not clear which are the most appropriate. ibid.

B. bacilliformis is the causative agent for Carrion's disease (Oroya fever), and is typically treated with chloramphenicol, penicillins or tetracyclines. ibid. Another species, B. elizabethae has been associated with cardiac valve abnormalities, and is so rare that appropriate antibiotics have yet to be determined. ibid.

B. quintana causes trench fever (rare except for unsanitary living conditions or in the immunocompromised), and has been successfully treated with penicillins, tetracyclines and cephalosporins. Kordick et al., 35(7) J. Clin. Microb. 1813 (1997). B. vinsonii sub vinsonii and B. vinsonii sub berkhoffii have only been found in dogs and voles.

Available Bartonella PCR diagnostics require several steps, and are therefore inconvenient for laboratory analysis of samples. For instance, PCR assays on the basis of differences in citrate synthase sequences have been performed using a first step of conducting PCR and a second step of digesting the PCR products with restriction enzymes, followed by gel electrophoresisis to distinguish among species. Joblet et al., 33(7) J. Clin. Microb. 1879 (1995); Norman et al., 33(7) J. Clin. Microb. 1797 (1995). PCR assays on the basis of differences in 16S rRNA sequences have also been conducted, using restriction enzymes to distinguish among species. Birtles, 129 FEMS Microbiol. Letters 261 (1995).

Likewise, primers have been used to amplify the region between the 16S and 23S genes (called “the intergenic region”) of Bartonella. In those assays, restriction enzymes were also used to cut and distinguish the PCR products. Matar et al., 31(7) J. Clin. Microb. 1730 (1993) and Roux and Raoult, 33(6) J. Clin. Microb. 1573 (1995). In Roux, a difference in size of PCR products (prior to digestion by restriction enzymes) was noted (page 1576); however, the differences are so small as to be indistinguishable on a gel. Moreover, no suggestion is made in Roux to use the pre-digestion PCR product size differences for the purpose of differentiation. In Matar, page 1732 that all three species had “an approximately 1,600-bp fragment” and bacilliformis had a 1,000 bp fragment prior to digestion.

In a different approach, Minnick and Barbian, 31 J Microb Meth 51 (1997) designed one set of primers from the 16S/23S intergenic region of Bartonella, and amplified fragments from B. bacilliformis, B. elizabethae, B. henselae and B. vinsonii. The fragments were of different, but indistinguishable sizes (FIG. 2), and the researchers therefore conducted a second, species-specific amplification using different sets of primers for each species represented (FIG. 3). Minnick, at 55 (1997).

Citation of the above documents is not intended as an admission that any of the foregoing is pertinent prior art. All statements as to the date or representation as to the contents of these documents is based on subjective characterization of information available to the applicant, and does not constitute any admission as to the accuracy of the dates or contents of these documents.

### SUMMARY OF THE INVENTION

The present invention requires only a single step to generate amplicons which identify a specific species.

It is therefore an object to provide a simplified assay for distinguishing between or among species of a genus.

It is a specific object to provide a simplified assay for distinguishing between or among Bartonella species.

It is yet another object to provide materials (nucleic acids, vectors, cells, etc.) related to the methods disclosed, including primers and the full sequence of the 16S/23S region of two species of Bartonella.

In all of the above embodiments, it is an object to provide methods to diagnose disease using the materials and methods provided.

It is also an object to provide methods for identifying primers useful to conduct PCR assays which capitalize on the species-specific size differences in an intergenic region of a prokaryote.

It is also an object to provide methods for identifying primers useful to conduct PCR assays which capitalize on the species-specific size differences in the 16S/23S intergenic region of Bartonella.

Finally, it is an object of the invention to provide a kit for convenient use of the materials and methods herein provided.

Definitions: For the purposes of the present invention, the following terms shall have the following meanings:

“Amplicon(s)” shall mean a nucleic acid(s) produced through use of primers in PCR.

“Genus-specific primer(s)” shall mean primers capable of amplifying an amplicon from at least a portion of the 16S/23S intergenic region of at least two species of the same genus, and no other genera, and wherein the size of the amplicon is unique to the species.

“Bartonella genus-specific primer(s)” shall mean primers capable of amplifying an amplicon from at least a portion of the 16S/23S intergenic region of at least two Bartonella species, and no other genera, and wherein the size of the amplicon is unique to the species.

When the term “Genus-specific primer(s)” or “Bartonella genus-specific primer(s)” is used to describe primers used in PCR assays, it is assumed that said primers are also being in amounts sufficient to amplify at least one ascertainable fragment.

A “set” of primers means at least one forward and at least one reverse primer, that, when used in a PCR assay in appropriate amounts, is capable of amplifying an amplicon.

### BRIEF DESCRIPTION OF THE FIGURES

FIG. 1. Nucleotide sequence alignment of a portion of the 16S-23S rRNA intergenic region of B. bacilliformis (GenBank accession #L26364), B. elizabethae (#L35103), B. henselae (#L35 101), B. quintana (#L35 100), and B. vinsonii (baker strain) (#L35102). Corresponding GenBank nucleotide numbers are indicated at the beginning and end of the sequences. Arrows designate PCR primer positions.

FIG. 2. PCR-based identification of Bartonella species. An ethidium bromide-stained agarose gel (3%) demonstrating amplified products from DNA template derived from Bartonella species. Bartonella bacilliformis, B. elizabethae, B. henselae, and B. quintana yielded expected products of 186 bp, 216 bp, 147 bp, and 132 bp, respectively. Template DNA from B. vinsonii (subspecies berkhoffii) yielded a PCR product of approximately 235 bp. PCR amplification of the B. clarridgeiae template DNA yielded no product. First and last lanes contain 20 base pair ladder.

FIG. 3. Phylogenetic comparison of 16S-23S rRNA intergenic region sequences for Bartonella species. Calculated matching percentages are indicated at each branch point of the dendrogram. The lengths of horizontal and vertical lines are not significant.

FIG. 4. Nucleotide sequence alignment of a portion of the 16S-23S rRNA intergenic region of B. bacilliformis (GenBank accession #L26364), B. clarridgeiae (GenBank accession #AF167989), B. elizabethae (#L35103), B. henselae (#L35101), B. quintana (#L35100), B. vinsonii (baker strain) (#L35102), and B. vinsonii (subspecies berkhoffii) (#AF167988). Corresponding GenBank nucleotide numbers are indicated at the beginning and end of the sequences. Arrows designate PCR primer positions. Non-conserved B. clarridgeiae nucleotide located at the 3′ end of initial PCR primer used (see FIG. 1) denoted by an asterisk (*).

FIG. 5. PCR-based identification of Bartonella species. An ethidium bromide-stained agarose gel (3%) demonstrating amplified products from DNA template derived from Bartonella species. Bartonella bacilliformis, B. clarridgeiae, B. elizabethae, B. henselae, B. quintana, and B. vinsonii (subspecies berkhoffii) yielded expected products of 211, 154 bp, 241 bp, 172 bp, 157 bp, and 260 bp, respectively. First and last lanes contain 20 base pair ladder.

FIG. 6. PCR-based identification of Bartonella species from animals known to be infected with either B. clarridgeiae (Sample A), B. henselae (Sample B), or B. vinsonii (subspecies berkhoffii) (Sample C). DNA was extracted from 2001 of blood, eluted in a final volume of 200 , l then 5 l of template DNA was used in each PCR amplification. After amplification, PCR products were electrophoresed on a 3% agarose gel and stained with ethidium bromide. Amplified control template DNA derived from isolated B. clarridgeiae, B. henselae, and B. vinsonii (subspecies berkhoffii) yielded expected products of 154 bp, 172 bp, and 260 bp, respectively. First and last lanes contain 20 base pair ladder.

### DETAILED DESCRIPTION OF THE INVENTION

In broad terms, the present invention includes materials and methods useful to distinguish between and among species of a genus. The methods simplify and are therefore more cost-effective than previous methods. In addition, because the present methods are simpler than previous methods, the risk of operator error, contamination, or any other technical problem is reduced, making the present invention inherently more reliable than previous methods.

The present invention includes methods to detect at least one prokaryotic species in a test sample, comprising the following steps: a.) conducting polymerase chain reaction using starting materials comprising a test sample and at least one set of genus-specific primers; and b.) detecting the prokaryotic species in the event that a species-specific sized amplicon is present. A method as described, wherein step b.) comprises gel electrophoresis is preferred, although any method for detecting amplicon(s) is within the scope of the present invention.

The present invention also includes methods to detect Bartonella species in a test sample, comprising: a.) conducting polymerase chain reaction using starting materials which comprise a test sample and at least one set of Bartonella genus-specific primers; and b.) detecting Bartonella species in the test sample in the event a Bartonella-sized amplicon is present. A method as described, wherein step b.) comprises gel electrophoresis is preferred, although any method for detecting amplicon(s), (e.g. size-differentiating chromatography) is within the scope of the present invention.

For instance, the above method can be used to identify both the specific presence, or the specific absence of a certain species of Bartonella. As an example, the present method could be used to test a sample using a primer set (for instance, one forward sequence, one reverse sequence, in amounts necessary to conduct PCR) designed to amplify, both B. bacilliformis and B. quintana, although the size of the amplicons would differ. In that instance, it is possible that the primers would amplify a fragment unique for B. quintana, and not B. bacilliformis. The result would indicate the presence of B. quintana as well as the absence of B. bacilliformis.

In another example, the present method could be used to test a sample using a primer set designed to amplify uniquely-sized amplicons from each and every known Bartonella species. Amplicons resulting from use of the genus-specific primer set would identify, by their size or absence, the species of Bartonella present and/or absent in the sample. For instance, if B. elizabethae and B. henselae-sized amplicons were present and B. quintana, B. bacilliformis and B. clarridgeiae-sized amplicons were absent, then the result would indicate the presence of B. elizabethae and B. henselae and the absence of B. quintana, B. bacilliformis and B. clarridgeiae. In fact, methods as described, wherein the primers are capable of amplifying uniquely-sized amplicons for every Bartonella species is a preferred embodiment of the present invention. However, methods wherein the primers are capable of amplifying uniquely-sized amplicons from B. henselae and B. clarridgeiae (the species which have been associated with cat scratch disease) are also preferred.

Moreover, the present invention is not limited to the use of only one set of genus-specific or Bartonella genus-specific primers. The methods herein also include those wherein a second set of primers is used, for example, for nested PCR. However, methods wherein PCR is conducted using one set of genus-specific or Bartonella genus-specific primers is preferred.

Methods which utilize primers designed using conserved sequences in or flanking the Bartonella 16S/23S intergenic region are within the scope of the present invention. The regions which span the consensus sequences in the Bartonella 16S/23S intergenic sequences from nucleotides 1-100, 130-150 and 300-350 (nucleotide numbers for B. henselae, Genbank Accession Number L35 101) are particularly useful for designing forward primers for the present methods. A preferred region for designing forward primers for the present invention is the region spanning bases 351 through 402. Not all bases are identical in these regions, but those in the art are aware of primer design strategy in light of non-identical sequences.

The regions which span the consensus sequences in the Bartonella 16S/23S intergenic sequences from nucleotides 430-530, 860-940 and 1000-1035 (nucleotide numbers for B. henselae, Genbank Accession Number L35101) are particularly useful for designing reverse primers for the present methods. A preferred region for designing reverse primers for the present invention is the region spanning bases 552 through 652. Not all bases are identical in these regions, but those in the art are aware of primer design strategy in light of non-identical sequences.

Methods as above wherein the Bartonella genus-specific forward primer is selected from the group consisting of: SEQ ID NO 5; SEQ ID NO 8; SEQ ID NO 9; SEQ ID NO 11; SEQ ID NO 14; SEQ ID NO 20; and SEQ ID NO 22 are preferred. More preferred are SEQ ID NO 5; SEQ ID NO 11; SEQ ID NO 14; SEQ ID NO 20; and SEQ ID NO 22. Methods as described in the previous paragraph wherein the Bartonella genus-specific reverse primer is selected from the group consisting of: SEQ ID NO 6; SEQ ID NO 7; SEQ ID NO 10; SEQ ID NO 12; SEQ ID NO 13; SEQ ID NO 19; and SEQ ID NO 21 are preferred. SEQ ID NO 6; SEQ ID NO 10; SEQ ID NO 13; SEQ ID NO 19; and SEQ ID NO 21 are more preferred. The most preferred forward primer for use in a diagnostic assay is SEQ ID NO 20, and the most preferred reverse primer for use in a diagnostic assay is SEQ ID NO 19.

Also provided in the present invention are methods to detect Bartonella-caused disease in a mammal, comprising: a.) conducting polymerase chain reaction using starting materials which comprise a test sample and at least one set of Bartonella genus-specific primers; and b.) detecting Bartonella-caused disease in the test sample in the event a Bartonella-sized amplicon is present. A method as in this paragraph, wherein the Bartonella-caused disease is bacilliary angiomatosis or cat scratch disease is preferred.

Specifically the present invention also provides methods to detect cat-scratch disease in a mammal, comprising: a.) conducting polymerase chain reaction using starting materials which comprise a test sample and at least one set of Bartonella genus-specific primers capable of amplifying B. henselae and/or B. clarridgeiae nucleic acid; and b.) detecting cat-scratch disease in the test sample in the event a B. henselae- and/or B. clarridgeiae-sized amplicon is present.

Despite the focus of the preceding paragraphs on the ability of the present invention to distinguish products from Bartonella species, the present invention is not limited thereto. The general concept of using intergenic sequences (or other variable regions) to conduct PCR so as to generate PCR products of distinguishable and distinguishing size is within the scope of the present invention. For example, the intergenic sequences, of certain Mycobacterium species are known to be variable, and primers common to the intergenic sequences of these organisms (and which would result in size-distinguishing products) would eliminate the extra step of having to conduct restriction enzyme digests on the PCR products. Moreover, even for those organisms for which an acceptable assay exists, the present invention is useful in that it easy and convenient to conduct.

Intergenic sequences of organisms are generally available through journal publications, or through Genbank or NIH blast database. The most used database can be found at http://www.ncbi.nlm.nih.gov/. A search for intergenic sequences would typically include searching on either a known sequence or the name of the organisms to be distinguished.

The primers for the above assay can be designed using the 16S/23S intergenic sequence from B. henselae (Genbank accession number L35101); B. bacilliformis (Genbank accession number L26364); B. quintana (Genbank accession number L35100); B. vinsonii sub vinsonii (Genbank accession number L35102); B. elizabethae (Genbank accession number L35103) and the sequence information herein provided. Moreover, it is known in the art that primers are preferrably G-C rich, ideally more than 50% of the bases being G or C. The length of the primer is usually chosen to minimize the chances of amplifying non-target nucleic acid, as well as minimize self-hybridization. Primers are typically 17 to 30 bases in length, although there are no absolute rules with regard to length or G-C content. For the purposes of the present invention, other parameters may take precedent over the length or constitution of the primers. Certain computer programs (such as MacVector) are helpful in primer design and PCR condition optimization.

The assays described herein comprise both a PCR step and an amplicon size-determination step. PCR can be conducted according to techniques known to those of skill in the art, including for example, thermocycle PCR and isothermal PCR. A number of printed publications describe these procedures. For instance Sambrook et al., Molecular Cloning: A Laboratory Manual (Cold Spring Harbor Laboratory Press, 1989); Ausubel et al., Current Protocols in Molecular Biology (Greene Publishing Associates, Inc., 1993); and Walker et al., 89 Proc Natl Acad Sci USA 392 (1992) describe typical parameters. Moreover, journal articles by investigators studying the organisms of interest will typically contain details about PCR amplification of the organisms' nucleic acid.

For example, thermocycle PCR can be conducted as follows: a sample is taken for amplification. Then, a thermocycler is used (at alternatingly high and low temperatures) to promote a.) dissociation of double stranded nucleic acid; and b.) hybridization of the primers to any sample nucleic acid and c.) subsequent synthesis of complementary nucleic acid. When the primers are bound to a nucleic acid in the test sample, the polymerase synthesizes a nucleic acid complementary to the sample nucleic acid, and when the primers are not bound, no synthesis takes place. A suitable biological sample includes, but is not limited to, a bodily fluid composition or a cellular composition. A bodily fluid refers to any fluid that can be collected (i.e., obtained) from an animal, examples of which include, but are not limited to, blood, serum, plasma, urine, tears, aqueous humor, cerebrospinal fluid (CSF), saliva, lymph, nasal secretions, milk and feces.

A second step in the described methods of the present invention is a size-determination of the PCR products generated. Size determination can be carried out according to any acceptable method, with gel electrophoresis being preferred. Methods for determining size of PCR products are described in Sambrook, supra and Ausubel, supra. Use of a control (identity known) sample or a sizing ladder is particularly helpful as well.

The present invention also includes kits useful for distinguishing between or among species of the same genus, comprising at least one set of genus-specific primers, said primers being capable of amplifying uniquely-sized fragments from at least a portion of an intergenic region of at least two species of said genus. The present kits preferably further comprise a gel material, such as, but not limited to, agarose or acrylamide.

Specifically, the present invention includes kits useful for distinguishing between or among Bartonella species, comprising at least one set of Bartonella genus-specific primers. The present kits preferably further comprise a gel material, such as, but not limited to, agarose or acrylamide.

Nucleic acid compounds are also provided by the present invention. Specifically, compositions of matter comprising forward and reverse Bartonella genus-specific primers as described herein are included in the present invention. Particular forward Bartonella genus-specific primers selected from the group consisting of: SEQ ID NO 5; SEQ ID NO 8; SEQ ID NO 9; SEQ ID NO 11; SEQ ID NO 14; SEQ ID NO 20; and SEQ ID NO 22 are included, with SEQ ID NO5; SEQ ID NO 11; SEQ ID NO 14; SEQ ID NO20; and SEQ ID NO22 being preferred. More preferred is SEQ ID NO 20. Particular reverse Bartonella genus-specific primers selected from the group consisting of: SEQ ID NO 6; SEQ ID NO 7; SEQ ID NO 10; SEQ ID NO 12; SEQ ID NO 13; SEQ ID NO 15; SEQ ID NO 19; and SEQ ID NO 21 are also included, with SEQ ID NO 6; SEQ ID NO 10; SEQ ID NO 13; SEQ ID NO 19; and SEQ ID NO 21 being preferred. More preferred is SEQ ID NO 19.

The sequences described in the sequence listing can be shortened from the 5′ end, provided that the resulting sequence does not result in loss of specificity when the shortened sequence is used as a primer. Those shortened primers are also useful as a part of a genus-specific primer set. For example, those primers wherein the 5′ terminus is shortened by 1-10 bases are also within the scope of the present invention. Primers wherein the 5′ terminus is shortened by 1-8 bases are preferred. Primers which are 14 bases in length and include at least one differentiating codon are most preferred. Any of these sequences can be used as primers in the methods described.

Also, with regard to the nucleic acid compounds herein provided, are isolated nucleic acid compounds comprising a 16S/23S intergenic sequence of Bartonella clarridgeiae, or a fragment thereof. Preferred nucleic acid compounds comprise SEQ ID NO 1, or a fragment thereof, and the antisense compound thereof, which is SEQ ID NO 2, or a fragment thereof. Moreover, vectors and cells comprising SEQ ID NO 1 and SEQ ID NO 2 or fragments thereof are also provided.

Isolated nucleic acid compounds comprising a 16S/23S intergenic sequence of Bartonella vinsonii, subspecies berkhoffii, or fragments thereof are also specifically provided. Preferred nucleic acid compounds comprise SEQ ID NO 3 and/or SEQ ID NO 23, or a fragment thereof, and the antisense compounds thereof, which are SEQ ID NO 4 and SEQ ID NO 24, respectively, or a fragment thereof. Moreover, vectors and cells comprising SEQ ID NO 3, SEQ ID NO 4, SEQ ID NO:23, and SEQ ID NO:24 or fragments thereof are also provided.

A “fragment” as used herein, is a nucleic acid molecule which is subset of the referent compound, and which is at least 12 bases in length. Preferably the fragments herein are at least about 20, 30, 40 or 50 or 100 bases in length.

The vectors of the present invention can be any vector, including those derived from prokaryotes, eukaryotes or viruses. The vectors can be synthetic or hybrids of any of the materials described. The regulatory regions can be any promoter, terminator, enhancer or other regulatory signal or sequence. Cells of the present invention can be any cells, including bacterial, fungal and/or eukaryotic cells.

Included within the scope of the present invention, with particular regard to the nucleic acids above, are allelic variants, degenerate sequences and homologues. By “nucleic acid(s)” is meant any allelic variant, hybrids, and fragments thereof. “Allelic variant” is meant to refer to a sequence that occurs at essentially the same locus (or loci) as ther referent sequence (eg. SEQ ID NOs 1 through 4), but which, due to natural variations caused by, for example, mutation or recombination, has a similar but not identical sequence. Allelic variants are well known to those skilled in the art and would be expected to be found within intergenic sequences. The present invention also includes variants due to laboratory manipulation, such as, but not limited to, variants produced during polymerase chain reaction amplification or site directed mutagenesis. A nucleic acid sequence homologous to a nucleic acid herein is characterized by the ability to hybridize to the exemplified nucleic acid compounds (or allelic variants or degenerates thereof) under stringent conditions. Stringent hybridization conditions are described in Sambrook, et al., Molecular Cloning: A Laboratory Manual, at 9.47-9.51 (Cold Spring Harbor Laboratory Press, 1989).

In another embodiment of the present invention, there are provided methods to detect Bartonella clarridgeiae in a test sample, comprising: conducting polymerase chain reaction using starting materials which comprise genus- or species-specific primers constructed from SEQ ID NO 1 and/or SEQ ID NO 2, under conditions which allow production of an amplicon in the event that Bartonella clarridgeiae is present in the test sample; and detecting Bartonella clarridgeiae in the event that an amplicon is present.

Additionally, there are provided methods to detect Bartonella vinsonii, subspecies berkhoffii in a test sample, comprising: conducting polymerase chain reaction using starting materials which comprise genus- or species-specific primers constructed from SEQ ID NO 3, SEQ ID NO 4, SEQ ID NO:23, and/or SEQ ID NO:24 under conditions which allow production of an amplicon in the event that Bartonella vinsonii, subspecies berkhoffii is present in the test sample; and detecting Bartonella vinsonii, subspecies berkhoffii in the event that an amplicon is present.

Lastly, the present invention includes methods to design a set of primers capable of amplifying a uniquely-sized fragment from at least two species of a genus, comprising: identifying genomic fragments from at least two species of the same genus, said genomic fragments having differences in absolute size, and said genomic fragments being defined at least on the periphery by alignable conserved sequences; and identifying a forward and reverse sequence on the periphery which are common to each of the species for which primers are desired.

### Examples

### Example 1

Comparison of 16S-23S rRNA Intergenic Sequences of Bartonella Species

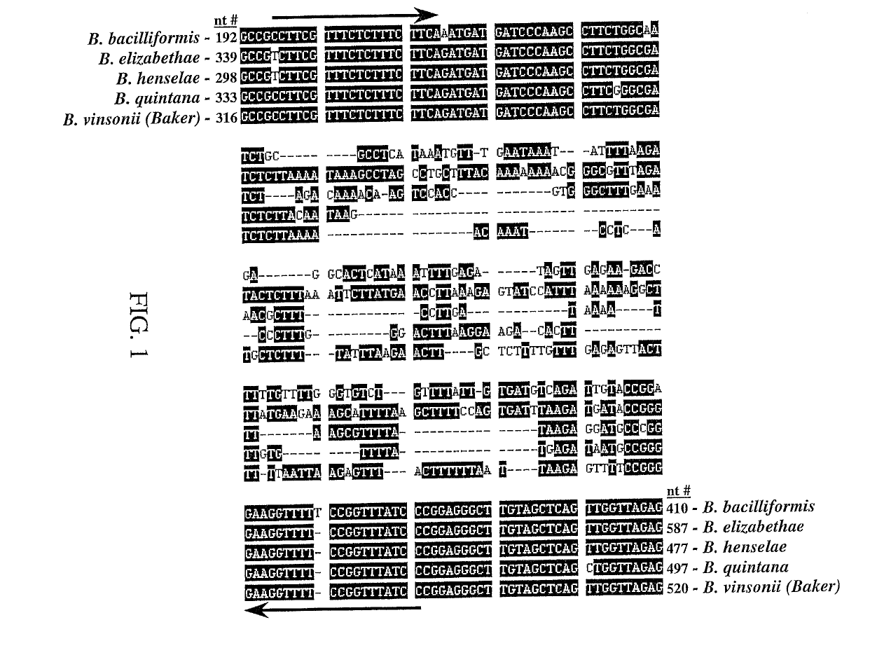

The 16S-23S rRNA intergenic sequences for B. bacilliformis (Genbank accession #L26364, 25), B. elizabethae (#L35103, 30), B. henselae (#L35101, 30), B. quintana (#L35100, 30), and B. vinsonii (baker strain) (#L35102, 30) were aligned using the DNA analysis computer program, DNAsis (Hitachi Software Engineering America Ltd., South San Francisco, Calif.). FIG. 1 illustrates alignment of approximately 200 nucleotides in the 5′ region of the 16S-23S intergenic sequences. In this region, a non-conserved area is bordered by two areas of high homology. Individual Bartonella species differ in the non-conserved region primarily due to sequence insertions and/or deletions. The extent of variation suggested that PCR primers designed to amplify across the non-conserved region would generate amplified products of different sizes for each species of Bartonella. A PCR assay was designed to amplify the region shown in FIG. 1. Template DNA was obtained from B. bacilliformis, B. clarridgeiae, B. elizabethae, B. henselae, B. quintana, and B. vinsonii (subspecies berkhoffii). B. koehlerae was not available for analysis at the time this work was performed. Further analysis of B. vinsonii (baker strain) was not included because this Bartonella species has not been associated with disease in either humans or domestic animals. Template DNA was amplified using 5′-(C/T)CTTCGTTTCTCTTTCTTCA-3′ (B. henselae nts 302-321, SEQ ID NO 14) and 5′-GGATAAACCGGAAAACCTTC-3′ (B. henselae nts 448-429, SEQ ID NO 7)) as forward and reverse primers, respectively. The 16S-23S rRNA intergenic sequences predict that these primers should amplify products of 186 bp (B. bacilliformis), 216 bp (B. elizabethae), 147 bp (B. henselae), and 132 bp (B. quintana). A predicted product size could not be determined for B. clarridgeiae or B. vinsonii (subspecies berkhoffii) because sequence of the 16S-23S rRNA intergenic region for these Bartonella species has not been reported.

### Example 2

Materials Used in PCR

Bacterial strains. B. bacilliformis (ATCC #35685), B. clarridgeiae (ATCC #51734 and #700095), B. elizabethae (ATCC #49927), B. quintana (ATCC #51694), B. vinsonii (subspecies berkhoffii) (ATCC #51572) were obtained from the American Type Culture Collection (Rockville, Md.). B. henselae isolates; Houston-1 (ATCC #49882), Oklahoma (ATCC #49793), Marseilles, MO-2, SA-1, CA-4, Tiger-2, and Lassiter were kindly provided by Russell Regnery, Viral and Rickettsial Diseases Branch, Centers for Disease Control and Prevention, Atlanta, Ga.

Clinical samples. Blood was obtained using aseptic procedures from the jugular vein of cats or dogs and placed in EDTA anti-coagulant tubes. Molecular characterization of B. henselae, B. clarridgeiae, and B. vinsonii (subspecies berkhoffii) isolates from these naturally-infected cats and dogs has been previously reported.

### Example 3

DNA Extraction and PCR Amplification of the 16S-23S rRNA Intergenic Region

DNA for PCR amplification was prepared from pure cultures of each bacterial strain using the QIAamp DNA Mini Kit (QIAGEN Inc., Valencia, Calif.) and from blood using the QIAamp DNA Blood Mini Kit. PCR amplifications were performed in 501 containing 10 mM Tris, pH 8.3, 50 mM KCl, 3.5 mM MgCl₂, 200 M each dATP, dCTP, and dGTP, 400 μM dUTP, 1 M each primer, and 2.5 units Amplitaq Gold DNA polymerase (PE Applied Biosystems, Foster City, Calif.). Amplification buffer was optimized with dUTP for use with Uracil glycosylase to prevent PCR amplification product carryover. Optimum primer annealing temperatures were determined in a RoboCycler® Gradient Temperature Cycler (Stratagene, La Jolla, Calif.). Amplifications were performed in a GeneAmp PCR System 9700 thermal cycler (PE Applied O Biosystems) using a time-release PCR protocol (13) as follows; 10 minute incubation at 20C. followed by 2 minutes denaturation at 95° C. then 45 cycles of 1 minute denaturation at 95° C., 1 minute annealing at 60° C., and 30 second extension at 72C. PCR amplification products were identified by ethidium bromide fluorescence after electrophoresis in 3% agarose gels.

### Example 4

Identifying Organisms Present

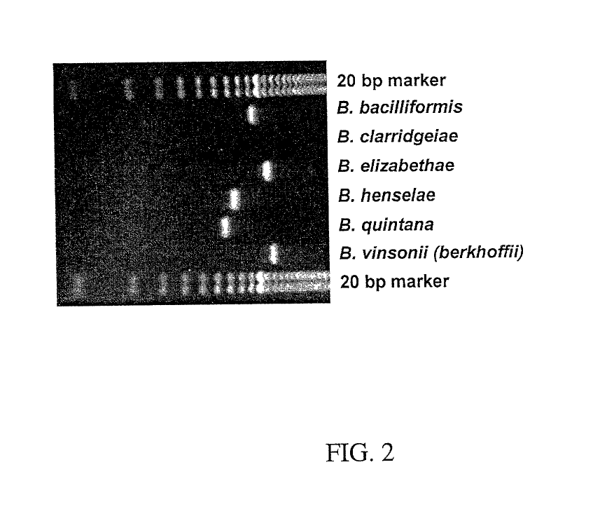

After amplification according to Example 3, PCR products were electrophoresed on a 3% agarose gel, stained with ethidium bromide and photographed. As shown in FIG. 2, the expected product size was amplified from B. bacilliformis, B. elizabethae, B. henselae, and B. quintana template DNA. The template DNA from B. vinsonii (subspecies berkhoffii) yielded a PCR product of approximately 235 bp rather than the 172 bp product predicted from the B. vinsonii (baker strain) sequence. PCR amplification of the B. clarridgeiae template DNA yielded no product using these primers.

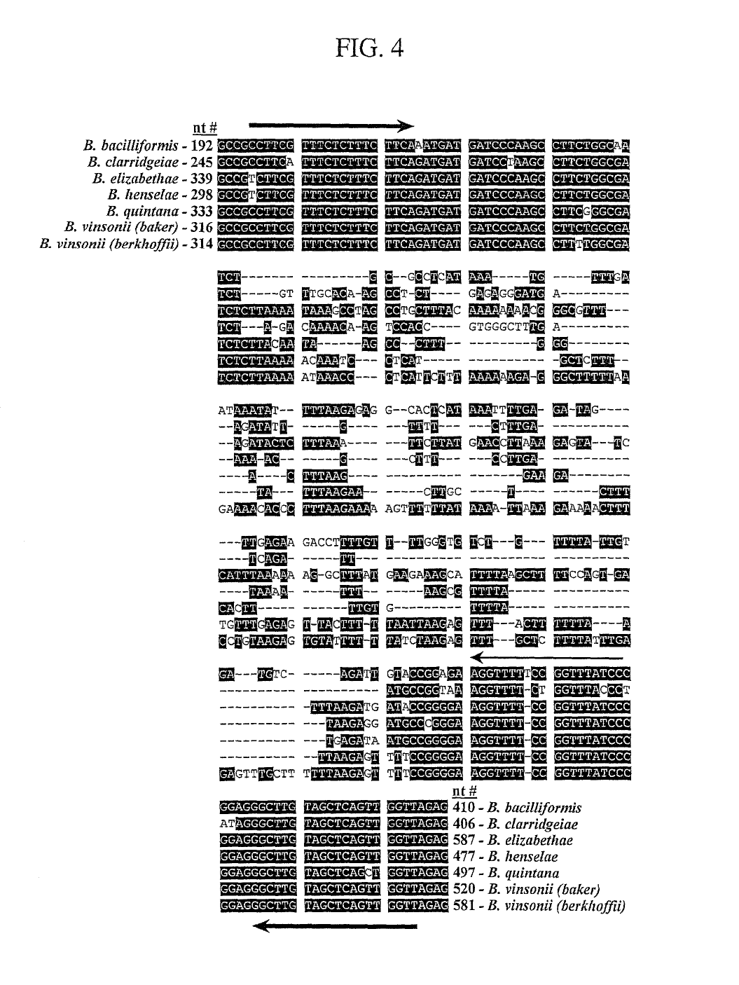

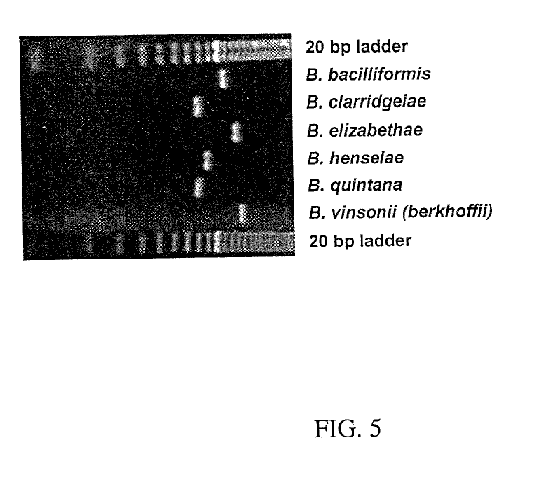

To detect and differentiate medically relevant Bartonella species, new primers complementary to 16S-23S rRNA intergenic region sequences shared by all of the Bartonella species were selected for PCR amplification (FIG. 4). Amplification of template DNA using 5′-(C/T)CTTCGTTTCTCTTTCTTCA-3′ (B. henselae nts 302-321, SEQ ID NO 14) and 5′-AACCAACTGAGCTACAAGCC-3′ (B. henselae nts 473-454, SEQ ID NO 19) as forward and reverse primers, respectively, resulted in amplified products corresponding to the predicted size, namely, 211 bp (B. bacilliformis), 154 bp (B. clarridgeiae), 241 bp (B. elizabethae), 172 bp (B. henselae), 157 bp (B. quintana), and 260 bp (B. vinsonii subspecies berkhoffii) (FIG. 5). Amplification of template DNA derived from the CA-4, MO-2, SA-1, Houston, Lassiter, Marseilles, Oklahoma, and Tiger-2 isolates of B. henselae yielded the same size amplification product, demonstrating conservation of this target region amongst different isolates of B. henselae (data not shown). Amplification of template DNA derived from Clostridium perfringens, Enterobacter cloacae, Escherichia coli, Ehrlichia canis, Ehrlichia chaffeensis, Ehrlichia equi, Ehrlichia equi, Ehrlichia ewingii, Ehrlichia risticii, Fusobacterium necrophorum, Klebsiella pneumoniae, Salmonella choleraesuis, and Staphylococcus intermedius did not result in product amplification (data not shown).

### Example 5

Sequencing of the 16S-23S rRNA Intergenic Region for B. clarridgeiae and B. vinsonii (subspecies berkhoffii)

PCR amplification of the entire 16S-23S rRNA intergenic region of the 16S-23S rRNA intergenic regions of B. clarridgeiae and B. vinsonii (subspecies berkhoffii) was accomplished using primers described by Matar et al and Roux et al. The resultant PCR products were sequenced using an ABI PRISM™ Model 377 with XL upgrade DNA Sequencer (PE Applied Biosystems) after product labeling using the PRISM™ Dye Terminator Cycle Sequencing Ready Reaction Kit (PE Applied Biosystems) following the manufacturer's protocol. The resultant sequence from B. clarridgeiae had a forward strand identified herein as SEQ ID NO: 1 and a reverse strand identified herein as SEQ ID NO:2. The resultant sequence from B. vinsonii (subspecies berkhoffii) had a forward strand identified herein as SEQ ID NO:3 and a reverse strand identified herein as SEQ ID NO:4.

An additional variant of the B. vinsonii (subspecies berkhoffii) 16S-23S rRNA intergenic region was identified as follows. DNA was extracted from a 200 μl sample of dog blood and PCR amplified using forward primer SEQ ID NO:20 and reverse primer SEQ ID NO: 19 under the following conditions: (A) a preliminary reaction consisting of 1 cycle of 20° C. for 10 minutes and 95° C. for 2 minutes; (B) 45 cycles of 95° C. for 1 minute, 60° C. for 1 minute, a seconds; and (C) 1 cycle of 72° C. for 10 minutes. The resultant PCR product was sequenced as described above to reveal a 216-nucleotide molecule determined to be a variant of the homologous region of the B. vinsonii (subspecies berkhoffii) sequence described above as SEQ ID NOs. 3 and 4. The variant, which represents a fragment of the 16S-23S rRNA intergenic region, corresponds to nucleotides 326-578 of SEQ ID NO:3 except that it contains a 36-nucleotide deletion corresponding to nucleotides 488-523 of SEQ ID NO:3 and two other nucleotide changes. The variant has a forward strand presented herein as SEQ ID NO:23 and a reverse strand presented herein as SEQ ID NO:24. Sequence alignments and phylogenetic comparisons were done with the DNA analysis computer program, DNAsis (Hitachi Software Engineering America Ltd., South San Francisco, Calif.).

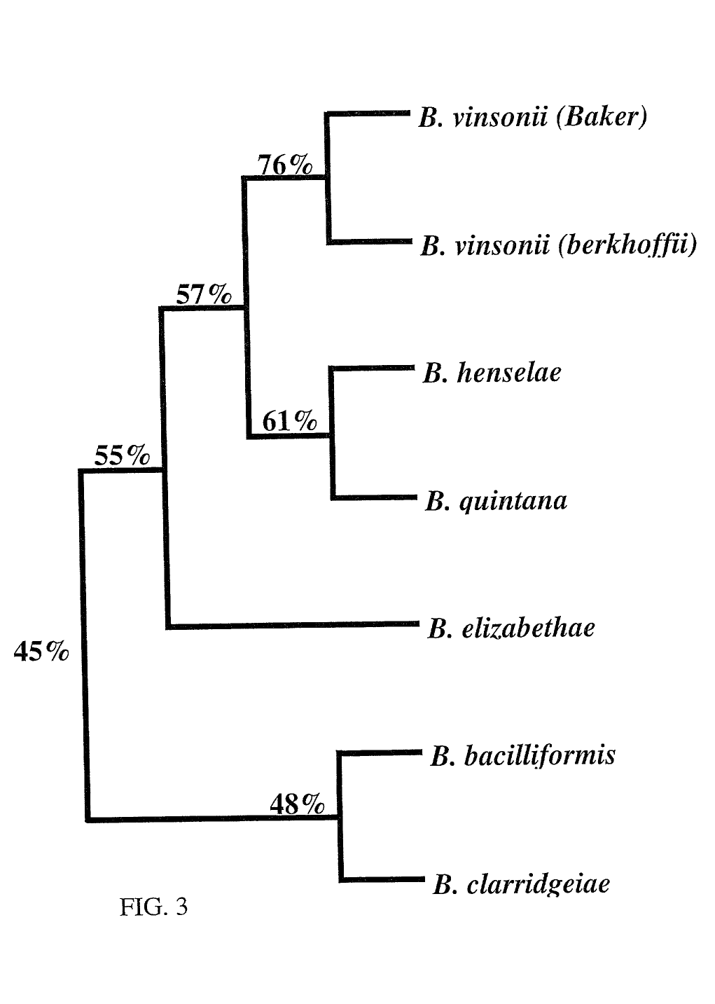

To investigate the failure to amplify product from B. clarridgeiae and the discrepancy between the amplification product from B. vinsonii (subspecies berkhoffii) and the size predicted from the B. vinsonii (baker strain) sequence, the 16S-23S rRNA intergenic regions from B. clarridgeiae and B. vinsonii (subspecies berkhoffii) were sequenced (Genbank #'s AF167989 and AF167988, respectively). The 16S-23S rRNA intergenic sequence of B. clarridgeiae and B. vinsonii (subspecies berkhoffii) were compared with the reported sequences of B. bacilliformis, B. elizabethae, B. henselae, B. quintana, and B. vinsonii (baker strain) (FIG. 3). As expected, B. vinsonii (subspecies berkhoffii) is most closely related to B. vinsonii (baker strain). Alignment of the B. vinsonii (subspecies berkhoffii) 16S-23S rRNA intergenic region revealed 63 bp inserted in the target region relative to the B. vinsonii (baker strain) sequence (FIG. 4). Based on 16S-23S rRNA intergenic region sequences, B. clarridgeiae is most closely related to B. bacilliformis (FIG. 3). Analysis of the B. clarridgeiae 16S-23S rRNA intergenic region sequence revealed that the 3′ nucleotide of the reverse PCR primer sequence is not conserved with other Bartonella species, thus explaining the inability to amplify a PCR product (FIG. 4). The 16S-23S rRNA intergenic sequences for B. clarridgeiae and B. vinsonii (subspecies berkhoffii), have been submitted to GenBank under accession no. AF167989 and AF167988, respectively

### Example 6.

PCR Detection in Clinical Samples

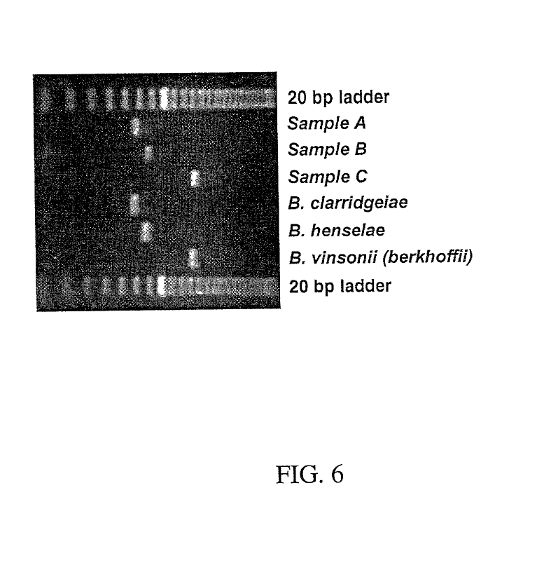

To evaluate the utility of this assay for detection of Bartonella species in clinical samples, DNA was prepared from blood of animals known to be infected with either B. henselae, B. clarridgeiae, or B. vinsonii (subspecies berkhoffii). Briefly, DNA was extracted from 200 1 of blood using the QIAamp® Blood Kit (QIAGEN Inc., Santa Clarita, Calif.) and eluted in a final volume of 200 1 per the manufacturer's protocol. Samples (5 1 of template DNA) were amplified using the primers described above. After amplification, PCR products were electrophoresed on a 3% agarose gel, stained with ethidium bromide and photographed. As illustrated in FIG. 6, the single-step PCR assay is capable of detecting and differentiating infections with B. henselae, B. clarridgeiae, and B. vinsonii (subspecies berkhoffii) in clinical samples derived from naturally-infected animals.

### Example 7.

Sensitivity of PCR Versus Blood Culture for Detection of B. henselae

To determine the sensitivity of the single-step PCR assay relative to blood culture we purified template DNA from 200 1 blood containing 10 to 100 CFU/ml of B. henselae derived from experimentally-infected cats. DNA was eluted in 200 l buffer and 5 l was used as template in the single-step PCR assay as described above. As shown in Table 1, the single-step PCR assay detected B. henselae in 100% of blood samples with 50-100 CFU/ml, 85% of blood samples with 30 CFU/ml, 75% of blood samples with 20 CFU/ml, and 75% of blood samples with 10 CFU/ml.

TABLE 1
Sensitivity of single-step PCR assay

| CFU's | Single-step PCR assay |
|---|---|
| 50-100 | 12/12 (100%) |
| 30 | 6/7 (85%) |
| 20 | 3/4 (75%) |
| 10 | 3/4 (75%) |

What is claimed is:

1. An isolated nucleic acid compound comprising a complete 16S/23S intergenic sequence of Bartonella clarridgeiae, or a fragment thereof.

2. A nucleic acid compound of claim 1, wherein the nucleic acid compound is selected from the group consisting of: SEQ ID NO 1; and SEQ ID NO 2.

3. A vector comprising the nucleic acid compound of claim 1.

4. A recombinant cell comprising the nucleic acid compound of claim 1.

5. An isolated nucleic acid compound comprising a complete 16S/23S intergenic sequence of Bartonella vinsonii, subspecies berkhoffii, or a fragment thereof.

6. A nucleic acid compound of claim 5, wherein the nucleic acid compound is selected from the group consisting of: SEQ ID NO 3; SEQ ID NO 4, SEQ ID NO:23, and SEQ ID NO:24.

7. A vector comprising the nucleic acid compound of claim 5.

8. A recombinant cell comprising the nucleic acid compound of claim 5.

9. A method to detect Bartonella clarridgeiae in a test sample, comprising:
conducting polymerase chain reaction using starting materials which comprise species-specific primers designed from a compound of claim 1, under conditions which allow production of an amplicon in the event that Bartonella clarridgeiae is present in the test sample; and
detecting Bartonella clarridgeiae in the event that an amplicon is present.

10. A method to detect Bartonella vinsonii, subspecies berkhoffii in a test sample, comprising:
conducting polymerase chain reaction using starting materials which comprise species-specific primers designed from a compound of claim 5, under conditions which allow production of an amplicon in the event that Bartonella vinsonii, subspecies berkhoffii is present in the test sample; and detecting Bartonella vinsonii, subspecies berkhoffii in the event that an amplicon is present.

11. A method to detect at least one prokaryotic species in a test sample, comprising:
a.) conducting polymerase chain reaction using starting materials which comprise at least one set of genus-specific primers; and
b.) detecting at least one of said species on the basis of amplicon size.

12. A method of claim 11, wherein step b.) comprises gel electrophoresis.


SEQUENCE LISTING (24 sequences)

SEQ ID NO: 1 - DNA, 1919 bp, Bartonella clarridgeiae
acaaggtagccgtaggggaacctgtggctggatcacctcctttctaaggatgatcaagaatgggcctaggccttttttgatctgattagacattgacggtttaaagtcttatttaaaccgttgacatattttaaacattctatgaaccgtgggttttgaatggaaactctgtcccctttagtgatacagagcataactgttttttatccatggttcatttgtttaaaaatttataaaaagactagccgccttcatttctctttcttcagatgatgatcctaagccttctggcgatctgtttgcacaagcctctgagagggatgaagatattgttttctttgatcagattatgccggtaaaggttttctggtttaccctatagggcttgtagctcagttggttagagcgcgcgcttgataagcgtgaggtcggaggttcaagtcctcccaggcccaccagttacacgatgctaaaagttgctatattgggagagttgataatcccttacaggaaattattgcccttaataaaactttattttctaaaagcattcagagctgacatagaatagagctgacatagaattgagaatctgacataggaattattgaaattgttttggaattattgaaattgttttctatcattttaaaaggctaaaatattctgtctctatttttaaaatagcatcaggtgttttgtaagagtgtgaagtttttaagtgtgaggttttttatattttagtgtgaggtttttataagggtatgacgtgagagcgttttgacctgttttaggggccgtagctcagctgggagagcacctgctttgcaagcagggggtcgtcggttcgatcccgtccggctccaccataatttggttcatcattattgttagaagaatagttattgcaagagattgagagatctctttgcttgttctattgaaattgtgaagaagaaggtatattcagacgttttttgcttgaactcattcttatgaaagagatttttcttatgaaagagatttttaagaatggatagcttaaaaagaagaatggatggcttaaaaaggtggcttaaaaaggatggctgtttttaaatgaaaatagttatttttacgctcttttgacgattgttacaacattatacgattaaaacattatacgataatgataataacgataataaaaagagctttcattaataaaaagagctttcattaataataaagagctttcattgaactttcattgaagaagcattttgagcaaaacagatgtgtcgcaaggaagagctcaaattccttgcttatgattggcaacttaaccgtgccattgaatatatctcgagaagttggtcttttctgctgatatttttgttttaagtgcctattgatgctagattatttttaaaaataattttgtattgatgattttgcacggaataattgacgaatgaatattggcaatgagaatgatcaagtgtcttaagggcatttggtggatgccttggcatgcacaggcgaagaaggacgtgatacgctgcgataagctacggggaggtgcgaataccctttgatccgtagatttccgaatggggcaacccaccttagattgctggaaaagttaagctgctttagattaaagcggtttaattttctagcaatcactaattaaggtatctgcatctgaataaaatagggtgtaagaagcgaacgcagggaactgaaacatctaagtacctgtaggaaaggacatcaatagagactccgttagtagtggcgagcgaacgcggaccaggccagtggcttaagttaagaaaagtagaatcgattggaaagtcgaaccaaagagggtgatagtcccgtatacgtagatctgatttaagtccttgagtaaggcgggacacgtgaaatcctgt

SEQ ID NO: 2 - DNA, 1919 bp, Bartonella clarridgeiae
acaggatttcacgtgtcccgccttactcaaggacttaaatcagatctacgtatacgggactatcaccctctttggttcgactttccaatcgattctacttttcttaacttaagccactggcctggtccgcgttcgctcgccactactaacggagtctctattgatgtcctttcctacaggtacttagatgtttcagttccctgcgttcgcttcttacaccctattttattcagatgcagataccttaattagtgattgctagaaaattaaaccgctttaatctaaagcagcttaacttttccagcaatctaaggtgggttgccccattcggaaatctacggatcaaagggtattcgcacctccccgtagcttatcgcagcgtatcacgtccttcttcgcctgtgcatgccaaggcatccaccaaatgcccttaagacacttgatcattctcattgccaatattcattcgtcaattattccgtgcaaaatcatcaatacaaaattatttttaaaaataatctagcatcaataggcacttaaaacaaaaatatcagcagaaaagaccaacttctcgagatatattcaatggcacggttaagttgccaatcataagcaaggaatttgagctcttccttgcgacacatctgttttgctcaaaatgcttcttcaatgaaagttcaatgaaagctctttattattaatgaaagctctttttattaatgaaagctctttttattatcgttattatcattatcgtataatgttttaatcgtataatgttgtaacaatcgtcaaaagagcgtaaaaataactattttcatttaaaaacagccatcctttttaagccacctttttaagccatccattcttctttttaagctatccattcttaaaaatctctttcataagaaaaatctctttcataagaatgagttcaagcaaaaaacgtctgaatataccttcttcttcacaatttcaatagaacaagcaaagagatctctcaatctcttgcaataactattcttctaacaataatgatgaaccaaattatggtggagccggacgggatcgaaccgacgaccccctgcttgcaaagcaggtgctctcccagctgagctacggcccctaaaacaggtcaaaacgctctcacgtcatacccttataaaaacctcacactaaaatataaaaaacctcacacttaaaaacttcacactcttacaaaacacctgatgctattttaaaaatagagacagaatattttagccttttaaaatgatagaaaacaatttcaataattccaaaacaatttcaataattcctatgtcagattctcaattctatgtcagctctattctatgtcagctctgaatgcttttagaaaataaagttttattaagggcaataatttcctgtaagggattatcaactctcccaatatagcaacttttagcatcgtgtaactggtgggcctgggaggacttgaacctccgacctcacgcttatcaagcgcgcgctctaaccaactgagctacaagccctatagggtaaaccagaaaacctttaccggcataatctgatcaaagaaaacaatatcttcatccctctcagaggcttgtgcaaacagatcgccagaaggcttaggatcatcatctgaagaaagagaaatgaaggcggctagtctttttataaatttttaaacaaatgaaccatggataaaaaacagttatgctctgtatcactaaaggggacagagtttccattcaaaacccacggttcatagaatgtttaaaatatgtcaacggtttaaataagactttaaaccgtcaatgtctaatcagatcaaaaaaggcctaggcccattcttgatcatccttagaaaggaggtgatccagccacaggttcccctacggctaccttgt

SEQ ID NO: 3 - DNA, 1883 bp, Bartonella vinsonii
gggcaggcaaccacggtagggtcagcgactggggtgaagtcgtaacaaggtagccgtaggggaacctgtggctggatcacctcctttctaaggatgatcaaaaattgggaaatcctctctttttgatctcatttagacacgggtttaaacttattcgtttaaaccgtgcatatgacactaacgctcaactctataccctctctcaaaagagaacctcttaacaaagaggccctaatctcttgaggcccctacctccgatgagatggtgttcatgagggcttagtgatcatgtgtaaaaataacaggcaggcttgccgccttcgtttctctttcttcagatgatgatcccaagccttttggcgatctcttaaaaataaaccctcattctttaaaaaagagggctttttaagaaaacaccctttaagaaaaagttttttataaaattaaagaaaaactttcctgtaagagtgtattttttatctaagagtttgctcttttatttgagagtttgcttttttaagagttttccggggaaggttttccggtttatcccggagggcttgtagctcagttggttagagcgcgcgcttgataagcgtgaggtcggaggttcaagtcctcccaggcccaccaatttagaattaccaattggaattgcttaacccactgttgagaaactccctcctttatgagagaaatctctaaaaacaagagaaagctcttgagagcctcaaatgatagatttcaaattctcaataagatttaaacccatcagatctaaatcaataaggtttaaaagtgttggtaaaatgttcataatatattttataaaaatattgagaattatggagatattaagagatttttcggacactattgataaatcttggtaagaaaattgataaaaatcggtttaggggttagcgctttcgtttaggggccgtagctcagctgggagagcacctgctttgcaagcagggggtcgtcggttcgatcccgtccggctccaccactttaggtcatcatcattgttatgagaaccgtctttagtgatgaggctttataacctttcgctcgttctattgaaattgtgaagagaagatatattcagacattgttcctttggggatgtttgttttgaaagaagcaagctttttgtgaagaaaaatgcttgtggtcaggaaaaacgcttcgttgaagaagaaacatcaaaagggtctctcaaagaaacccaagaaagaaaccaagaaacattgtgtcgcaagggaatgctcaaagcccttgcttatgattggaagcttaaccgcgccattgaatatatctcgagaagctggtcttttctgctgatattttgtttgttttgccctttttgttttgcacaaggcaaaaaaagagacaaacgctgaatgaatattggcaatgagaacgatcaagtgtcttaagggcatttggtggatgccttggcatgcacaggcgatgaaggacgtgatacgctgcgataagctacggggaggtgcgaataccctttgatccgtagatctccgaatggggcaacccacctttgatggctagaaaaattaagctgtttttacaaaaagacagtttagttttctagtcgtcagataaaggtatctacacctgaataaaatagggtgtaagaagcaaacgcagggaactgaaacatctaagtacctgtaggaaaggacatcaaacgagactccgttagtagtggcgagcgaacgcggaccaggccagtggcttaaattaagaaaagtagaatcgactggaaagtcgaaccaaagtgggtgatagtcccgtatacgtaaatctgatttaagtcctagagtagggcgggacacgtgaaatcctgtctgaatatgggt

SEQ ID NO: 4 - DNA, 1883 bp, Bartonella vinsonii
acccatattcagacaggatttcacgtgtcccgccctactctaggacttaaatcagatttacgtatacgggactatcacccactttggttcgactttccagtcgattctacttttcttaatttaagccactggcctggtccgcgttcgctcgccactactaacggagtctcgtttgatgtcctttcctacaggtacttagatgtttcagttccctgcgtttgcttcttacaccctattttattcaggtgtagatacctttatctgacgactagaaaactaaactgtctttttgtaaaaacagcttaatttttctagccatcaaaggtgggttgccccattcggagatctacggatcaaagggtattcgcacctccccgtagcttatcgcagcgtatcacgtccttcatcgcctgtgcatgccaaggcatccaccaaatgcccttaagacacttgatcgttctcattgccaatattcattcagcgtttgtctctttttttgccttgtgcaaaacaaaaagggcaaaacaaacaaaatatcagcagaaaagaccagcttctcgagatatattcaatggcgcggttaagcttccaatcataagcaagggctttgagcattcccttgcgacacaatgtttcttggtttctttcttgggtttctttgagagacccttttgatgtttcttcttcaacgaagcgtttttcctgaccacaagcatttttcttcacaaaaagcttgcttctttcaaaacaaacatccccaaaggaacaatgtctgaatatatcttctcttcacaatttcaatagaacgagcgaaaggttataaagcctcatcactaaagacggttctcataacaatgatgatgacctaaagtggtggagccggacgggatcgaaccgacgaccccctgcttgcaaagcaggtgctctcccagctgagctacggcccctaaacgaaagcgctaacccctaaaccgatttttatcaattttcttaccaagatttatcaatagtgtccgaaaaatctcttaatatctccataattctcaatatttttataaaatatattatgaacattttaccaacacttttaaaccttattgatttagatctgatgggtttaaatcttattgagaatttgaaatctatcatttgaggctctcaagagctttctcttgtttttagagatttctctcataaaggagggagtttctcaacagtgggttaagcaattccaattggtaattctaaattggtgggcctgggaggacttgaacctccgacctcacgcttatcaagcgcgcgctctaaccaactgagctacaagccctccgggataaaccggaaaaccttccccggaaaactcttaaaaaagcaaactctcaaataaaagagcaaactcttagataaaaaatacactcttacaggaaagtttttctttaattttataaaaaactttttcttaaagggtgttttcttaaaaagccctcttttttaaagaatgagggtttatttttaagagatcgccaaaaggcttgggatcatcatctgaagaaagagaaacgaaggcggcaagcctgcctgttatttttacacatgatcactaagccctcatgaacaccatctcatcggaggtaggggcctcaagagattagggcctctttgttaagaggttctcttttgagagagggtatagagttgagcgttagtgtcatatgcacggtttaaacgaataagtttaaacccgtgtctaaatgagatcaaaaagagaggatttcccaatttttgatcatccttagaaaggaggtgatccagccacaggttcccctacggctaccttgttacgacttcaccccagtcgctgaccctaccgtggttgcctgccc

SEQ ID NO: 5 - DNA, 20 bp, Artificial sequence
  Synthetic Primer A7518H09
gctggatcacctcctttcta

SEQ ID NO: 6 - DNA, 20 bp, Artificial sequence
  Synthetic Primer A7518H10
tccgacctcacgcttatcaa

SEQ ID NO: 7 - DNA, 20 bp, Artificial sequence
  Synthetic Primer A7518H12
ggataaaccggaaaaccttc

SEQ ID NO: 8 - DNA, 20 bp, Artificial sequence
  Synthetic Primer A7620C06
gatgatgatcccaagccttc

SEQ ID NO: 9 - DNA, 18 bp, Artificial sequence
  Synthetic Primer A8410C08
cgtaggggaacctgtggc

SEQ ID NO: 10 - DNA, 18 bp, Artificial sequence
  Synthetic Primer A8410C09
aacctccgacctcacgct

SEQ ID NO: 11 - DNA, 18 bp, Artificial sequence
  Synthetic Primer A8410C10
ctttcttcagatgatgat

SEQ ID NO: 12 - DNA, 17 bp, Artificial sequence
  Synthetic Primer A8450A02
ataaaccggaaaacctt

SEQ ID NO: 13 - DNA, 18 bp, Artificial sequence
  Synthetic Primer A8410C11
taaccaactgagctacaa

SEQ ID NO: 14 - DNA, 20 bp, Artificial sequence
  Synthetic Primer A7518H11
tcttcgtttctctttcttca

SEQ ID NO: 15 - DNA, 20 bp, Artificial sequence
  Synthetic Primer A8120F10
tactggttcactatcggtca

SEQ ID NO: 16 - DNA, 15 bp, Artificial sequence
  Synthetic Primer A8120F11
gccaaggcatccacc

SEQ ID NO: 17 - DNA, 16 bp, Artificial sequence
  Synthetic Primer A8744A03
aagtcgtaacaaggta

SEQ ID NO: 18 - DNA, 20 bp, Artificial sequence
  Synthetic Primer A8744A04
agaggcaggcaaccacggta

SEQ ID NO: 19 - DNA, 20 bp, Artificial sequence
  Synthetic Primer Z5610H12
aaccaactgagctacaagcc

SEQ ID NO: 20 - DNA, 22 bp, Artificial sequence
  Synthetic Primer
ctctttcttcagatgatgatcc

SEQ ID NO: 21 - DNA, 20 bp, Artificial sequence
  Synthetic Primer
taaccaactgagctacaagc

SEQ ID NO: 22 - DNA, 25 bp, Artificial sequence
  Synthetic Primer
tttctctttcttcagatgatgatcc

SEQ ID NO: 23 - DNA, 216 bp, Bartonella vinsonii
ctctttcttcagatgatgatcccaagccttttggcgatctcttaaaaacaaaccctcattctttaaaaaagagggctttttaagaaaacaccctttaagaaaaagttttttataaaattaaagaaaaactttcttgtaagagtgtattttttatctaagagttttccggggaaggttttccggtttatcccggagggcttgtagctcagttggtta

SEQ ID NO: 24 - DNA, 216 bp, Bartonella vinsonii
taaccaactgagctacaagccctccgggataaaccggaaaaccttccccggaaaactcttagataaaaaatacactcttacaagaaagtttttctttaattttataaaaaactttttcttaaagggtgttttcttaaaaagccctcttttttaaagaatgagggtttgtttttaagagatcgccaaaaggcttgggatcatcatctgaagaaagag